(19) United States
(12) Patent Application Publication
(10) Pub. No.: US 2008/0294088 A1
(43) Pub. Date: Nov. 27, 2008

(54) Biodegradable Tissue Cutting Device, A Kit And A Method For Treatment Of Disorders In The Heart Rhythm Regulation System

(76) Inventors: Jan Otto Solem, Stetten (CH); Stevan Nielsen, Rottenburg Am Neckar (DE); Ib Joergensen, Haigerloch (DE); Gerd Siebold, Ammerbuch (DE); Bodo Quint, Rottenburg (DE)

Correspondence Address: INSKEEP INTELLECTUAL PROPERTY GROUP, INC, 2281 W. 190TH STREET, SUITE 200, TORRANCE, CA, 90504, US

(21) Appl. No.: 11/910,607

(22) PCT Filed: May 17, 2006
(86) PCT No.: PCT/EP2006/062400

§ 371 Date: Jul. 22, 2008

Related U.S. Application Data
(60) Provisional application No. 60/303,573, filed on Jul. 6, 2001.
(60) Provisional application No. 60/451,865, filed on Mar. 3, 2003.
(60) Provisional application No. 60/451,864, filed on Mar. 3, 2003.

(51) Int. Cl. A61B 17/32

(52) U.S. Cl. 604/22; 606/159

(57) ABSTRACT

A tissue cutting device is disclosed, which is structured and arranged to be inserted through the vascular system into a body vessel adjacent to the heart and/or into the heart, and to be subsequently subjected to a change of shape in order to penetrate into the heart tissue. The tissue cutting device may thus be used for treating disorders to the heart rhythm regulation system. A kit of tissue cutting devices provides a plurality of devices for creating a lesion pattern for treating such disorders. The tissue cutting device is of a biodegradable material, such as hydrolytically degradable material or an enzymatically degradable material.

### RELATED APPLICATIONS

This application claims priority to International Patent Application No. PCT/EP2006/062400 filed May 17, 2006 entitled A Biodegradable Tissue Cutting Device, A Kit And A Method For Treatment Of Disorders In The Heart Rhythm Regulation System, which claims priority to International Patent Application No. PCT/EP2005/005363 filed May 17, 2005 entitled A Device And Kit For Treatment Of Disorders In The Heart Rhythm Regulation System, both of which are hereby incorporated by reference.

### FIELD OF THE INVENTION

The present invention relates to treatment of disorders in the heart rhythm regulation system and, specifically, to a tissue cutting device, a kit of shape-changing devices and a method for treating such disorders.

### BACKGROUND OF THE INVENTION

The circulation of blood in the body is controlled by the pumping action of the heart. The heart expands and contracts by the force of the heart muscle under impulses from the heart rhythm regulation system. The heart rhythm regulation system transfers an electrical signal for activating the heart muscle cells.

The normal conduction of electrical impulses through the heart starts in the sinoatrial node, travels across the right atrium, the atrioventricular node, the bundles of His and thereafter spread across the ventricular muscle mass. Eventually when the signal reaches the myocytes specialized in only contraction, the muscle cell will contract and create the pumping function of the heart (see FIG. 1).

The electrical impulses are transferred by specially adapted cells. Such a cell will create and discharge a potential over the cell membrane by pumping ions in and out of the cell. Adjacent cells are joined end-to-end by intercalated disks. These disks are cell membranes with a very low electrical impedance. An activation of a potential in a cell will propagate to adjacent cells thanks to the low impedance of the intercalated disks between the cells. While being at the embryonic stage, all heart muscle cells, the myocytes, have the ability to create and transfer electrical signals. During evolution the myocytes specialize and only those cells necessary for maintaining a stable heart-rate are keeping the ability to create and send electrical impulses. For a more thorough explanation of the propagation of electrical signals in the heart, see e.g. Sandoe, E. and Sigurd, B., Arrhythmia, Diagnosis and Management, A Clinical Electrocardiographic Guide, Fachmed AG, 1984.

The heart function will be impaired if there is a disturbance on the normal conduction of the electrical impulses. Atrial fibrillation (AF) is a condition of electrical disorder in the heart rhythm regulation system. In this condition, premature and fast signals irregularly initiating muscle contractions in the atria as well as in the ventricles will be started in ectopic sites, that is areas outside the sinoatrial node. These signals will be transmitted erratically all over the heart. When more than one such ectopic site starts to transmit, the situation becomes totally chaotic, in contrast to the perfect regularity in a healthy heart, where the rhythm is controlled from the sinoatrial node.

Atrial fibrillation is a very common disorder, thus 5% of all patients that undergo heart surgery suffer from AF. 0.4-2% of a population will suffer from AF, whereas 10% of the population over the age of 65 suffers from AF. 160 000 new cases occur every year in the US and the number of cases at present in the US is estimated to be around 3 million persons. Thus, treatment of atrial fibrillation is an important topic.

Typical sites for ectopic premature signals in AF may be anywhere in the atria, in the pulmonary veins (PV), in the coronary sinus (CS), in the superior vena cava (SVC) or in the inferior vena cava (IVC). There are myocardial muscle sleeves present around the orifices and inside the SVC, IVC, CS and the PVs. Especially around the orifice of the left superior pulmonary vein (LSPV) such ectopic sites are frequent, as well as at the orifice of the right superior pulmonary vein (RSPV). In AF multiple small circles of a transmitted electrical signal started in an ectopic site may develop, creating re-entry of the signal in circles and the circle areas will sustain themselves for long time. There may be only one ectopic site sending out signals leading to atrial flutter, or there may be multiple sites of excitation resulting in atrial fibrillation. The conditions may be chronic or continuous since they never stop. In other cases there may be periods of normal regular sinus rhythm between arrhythmias. The condition will then be described as intermittent.

In the chronic or continuous cases, the atrial musculature undergoes an electrical remodelling so that the re-entrant circuits sustain themselves continuously. The patient will feel discomfort by the irregular heart rate, sometimes in form of cannon waves of blood being pushed backwards in the venous system, when the atria contract against a closed arterio-ventricle valve. The irregular action of the atria creates standstill of blood in certain areas of the heart, predominantly in the auricles of the left and right atrium. Here, blood clots may develop. Such blood clots may in the left side of the heart get loose and be taken by the blood stream to the brain, where it creates disastrous damage in form of cerebral stroke. AF is considered to be a major cause of stroke, which is one of the biggest medical problems today.

Today, there are a few methods of treating the problems of disorders to the heart rhythm regulation system. Numerous drugs have been developed to treat AF, but the use of drugs is not effective to a large part of the patients. Thus, there has also been developed a number of surgical therapies.

Surgical therapy was introduced by Drs. Cox, Boineau and others in the late 1980s. The principle for surgical treatment is to cut all the way through the atrial wall by means of knife and scissors and create a total separation of the tissue. Subsequently the tissues are sewn together again to heal by fibrous tissue, which does not have the ability to transmit myocardial electrical signals. A pattern of cutting was created to prohibit the propagation of impulses and thereby isolate the ectopic sites, and thus maintain the heart in sinus rhythm. The rationale for this treatment is understandable from the description above, explaining that there must be a physical contact from myocyte to myocyte for a transfer of information between them. By making a complete division of tissue, a replacement by non-conductive tissue will prohibit further ectopic sites to take over the stimulation. The ectopic sites will thus be isolated and the impulses started in the ectopic sites will therefore not propagate to other parts of the heart.

It is necessary to literally cut the atria and the SVC and the IVC in strips. When the strips are sewn together they will give the impression of a labyrinth guiding the impulse from the sinoatrial node to the atrioventricular node, and the operation was consequently given the name Maze. The cutting pattern is illustrated in FIG. 2 and was originally presented in J L Cox, TE Canavan, RB Schuessler, M E Cain, BD Lindsay, C Stone, PK Smith, PB Corr, and JP Boineau, The surgical treatment of atrial fibrillation. II. Intraoperative electrophysiologic mapping and description of the electrophysiologic basis of atrial flutter and atrial fibrillation, J Thorac Cardiovasc Surg, 1991 101: 406-426. The operation has a long-time success of curing patients from AF in 90% of the patients. However, the Maze operation implicate that many suture lines have to be made and requires that the cuts are completely sealed, which is a demanding task for every surgeon that tries the method. The operation is time consuming, especially the time when the patients own circulation has to be stopped and replaced by extracorporeal circulation by means of a heart-lung machine. Thus mortality has been high and the really good results remained in the hands of a few very trained and gifted surgeons.

The original Maze operation has therefore been simplified by eliminating the number of incisions to a minimum, still resulting in a good result in most cases. The currently most commonly used pattern of incisions is called Maze III (see FIG. 3).

Other methods of isolating the ectopic sites have also been developed recently. In these methods, the actual cutting and sewing of tissue has been replaced by methods for killing myocyte cells. Thus, one may avoid separating the tissue, instead one destroy the tissue by means of heat or cooling in the Maze pattern to create a lesion through the heart wall. The damaged myocyte tissue can not transfer signals any more and therefore the same result may be achieved. Still the chest has to be opened, and the heart stopped and opened. Further, the energy source has to be carefully controlled to affect only tissue that is to be destroyed.

A large number of devices have now been developed using various energy sources for destroying the myocyte tissue. Such devices may use high radio frequency energy, as disclosed in e.g. U.S. Pat. No. 5,938,660, or microwaves, ultrasound or laser energy. Recently, devices have been developed for catheter-based delivery of high radio frequency energy through the venous and or arterial systems. However, this has so far had limited success due to difficulties in navigation and application of energy and also late PV stenosis has been reported. Further, devices using cooling of tissue has used expanding argon gas or helium gas to create temperatures of −160° C. Using an instrument with a tip, tissue can be frozen and destroyed.

WO 03/003948 discloses an apparatus for treating, preventing, and terminating arrhythmias. The device, which is implanted and left at the target site, is provided with protrusions that pierce the tissue, via self-expansion or balloon expansion, to gain access to the cells of said target site. The protrusions are used to conduct drugs to the cells, which drugs may cause cell death to thereby induce cellular changes that may lead to treatment of arrhythmias. Nowhere in WO 03/003948 is a device described that by expansion fully penetrates the wall of the blood vessel to disrupt cardiac impulses, which device then is bio-absorbed and thereby eliminated from the target site. The device according to WO 03/003948 is not a cutting device. The device according to WO 03/003948 therefore has to be removed from the site of action, to ensure that it does not harm, or in any other way inflict negatively with, tissue in the vicinity of the site of action. Furthermore, to affect function mechanisms, the devices according to prior art has to be supplied with other active substances, which active substances may be released at a preferred treatment site. Thus, an additional supply step has to be integrated in the manufacturing of such a device.

### OBJECTS AND SUMMARY OF THE INVENTION

Accordingly, the present invention seeks to mitigate, alleviate or eliminate one or more of the above-identified deficiencies and to provide a new device, and kit of devices, suitable for a method for treatment of disorders to the heart rhythm regulation system of the kinds referred to, according to the appended independent claims.

For this purpose a tissue cutting device according to claim 1 is provided, wherein the device is structured and arranged to be inserted in a temporary delivery shape through the vascular system into a body vessel adjacent to the heart and/or into the heart and to be subsequently subjected to a change of shape, from said temporary delivery shape via an expanded delivered shape to a further expanded shape, extending at least beyond an inner surface of said tissue, in order to create cutting action configured for cutting said heart tissue and/or said body vessel, wherein said cutting device is biodegradable.

Advantageous features of the invention are defined in the dependent claims.

### BRIEF DESCRIPTION OF THE DRAWINGS

The invention will now be described in further detail by way of example under reference to the accompanying drawings, on which:

FIG. 1 is a schematic view of the transmission of electrical signals in the heart;

FIG. 2 is a schematic view of a pattern of cutting tissue of the heart wall according to the Maze-procedure for treating disorders to the heart rhythm regulation system;

FIG. 3 is a schematic view of a simplified pattern according to the Maze III-procedure, wherein the heart is seen from behind;

FIGS. 4a-4c are perspective schematic views of a tissue cutting device according to an embodiment of the invention, wherein FIG. 4a shows the tissue cutting device in a first, temporary shape, FIG. 4b shows the tissue cutting device in a second, permanent shape, and FIG. 4c illustrates the tissue cutting device having sharp edges; and

FIGS. 5a-5b show the tissue cutting device of FIGS. 4a-4b inserted in a body vessel.

### DETAILED DESCRIPTION OF THE INVENTION

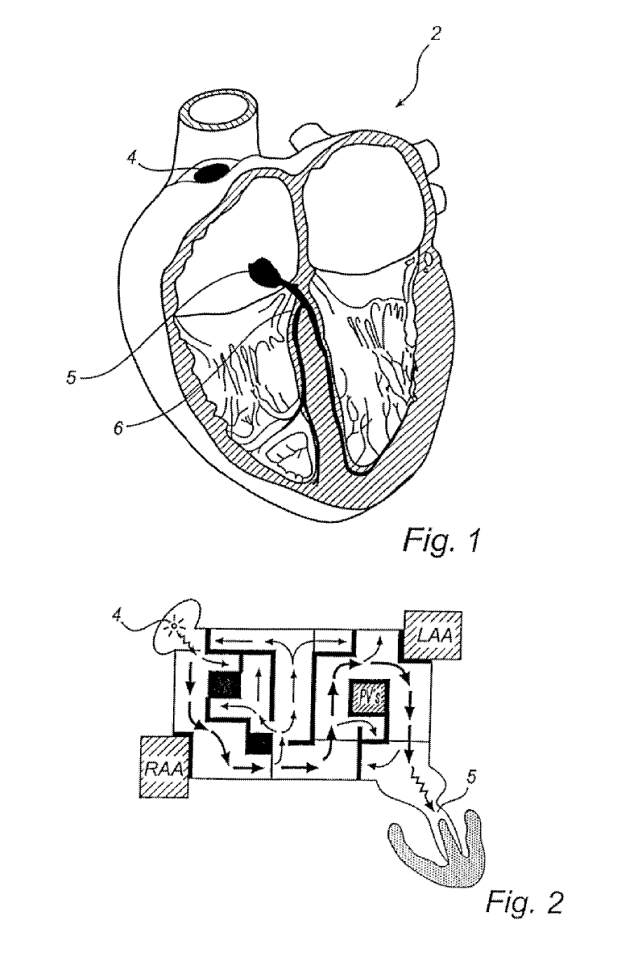

Referring now to FIGS. 1-3, the problems of disorders to the heart rhythm regulation system and the leading current method of treating these problems will be described. In FIG. 1, a heart 2 is shown and the controlling of the heart rhythm is indicated. The heart rhythm is normally controlled from the sinoatrial node 4. The sinoatrial node 4 transmits electrical signals which are propagated through the heart wall by means of special cells forming an electrical pathway. The electrical signals following the electrical pathway will coordinate the heart muscle cells for almost simultaneous and coordinated contraction of the cells in a heart atrium and heart ventricle. The normal conduction of electrical impulses through the heart starts in the sinoatrial node 4, travels across the right atrium, the atrioventricular node 5, the bundles of His 6 and thereafter spread across the ventricular muscle mass. In a disordered situation, electrical signals are started in heart cells outside the sinoatrial node 4, in so called ectopic sites. These electrical signals will disturb the coordination of the heart muscle cells. If several ectopic sites are present, the signal transmission becomes chaotic. This will be the cause of arrhythmic diseases, such as atrial fibrillation and atrial flutter.

An existing method for treating these diseases is based on isolating the ectopic sites in order to prevent the electrical signals started in these ectopic sites to propagate in the heart wall. Thus, the heart wall is cut completely through for interrupting the coupling between cells that transmit erratic electrical signals. The thus created lesion will be healed with fibrous tissue, which is unable to transmit electrical signals. Thus, the path of the electrical signals is blocked by this lesion. However, since the location of the ectopic sites may not always be known and may be difficult to determine or since there might be multiple ectopic sites, a special cutting pattern has been developed, which will effectively isolate ectopic sites. Thus, the same pattern may always be used regardless of the specific locations of the ectopic sites in each individual case. The procedure is called the “Maze”-procedure in view of the complicated cutting pattern. In FIG. 2, the Maze-pattern is illustrated.

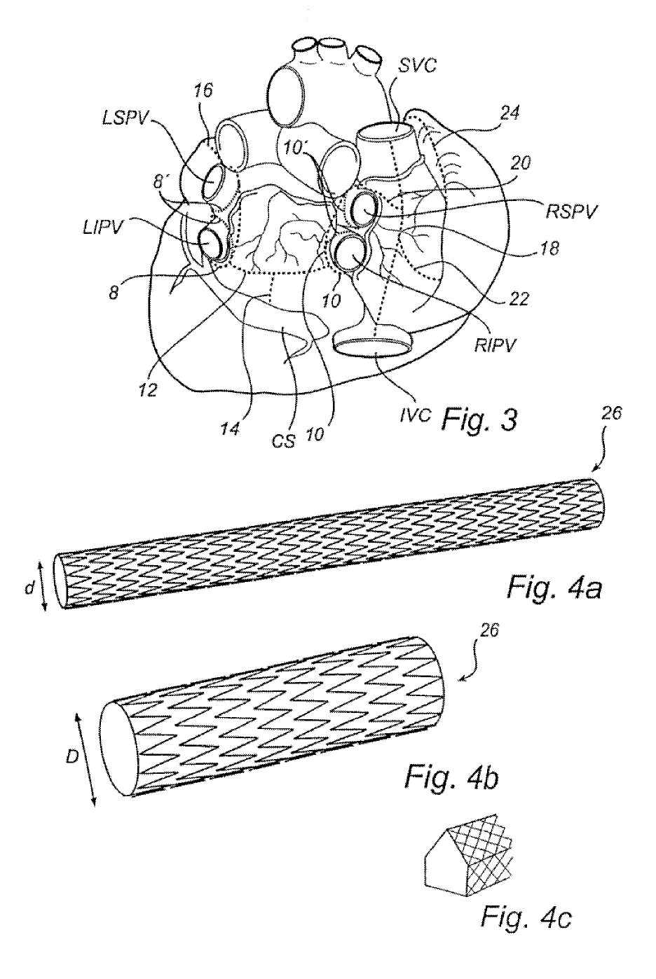

However, as is evident from FIG. 2, the cutting pattern is extensive and complex and requires a difficult surgery. Thus, the Maze-pattern has been evolved in order to minimize the required cuttings and simplify the pattern as much as possible. Currently, a Maze III-pattern is used, as shown in FIG. 3. This pattern is not as complicated, but would still effectively isolate the ectopic sites in most cases. The Maze III-pattern comprises a cut 8 around the left superior pulmonary vein (LSPV) and the left inferior pulmonary vein (LIPV) and a corresponding cut 10 around the right superior pulmonary vein (RSPV) and the right inferior pulmonary vein (RSPV); a cut 12 connecting the two cuts 8 and 10 around the pulmonary veins (PV); a cut 14 from this connecting cut to the coronary sinus (CS); a cut 16 from the left PVs to the left atrial appendage; a cut 18 from the inferior vena cava (IVC) to the superior vena cava (SVC); a cut 20 connecting the cut 10 around the right PVs and the cut 18 between the IVC and the SVC; a cut 22 from the cut 18 between the IVC and the SVC along the right lateral atrium wall; and a cut 24 isolating the right atrial appendage. Thus, a pattern, which is less complex and which effectively isolates the ectopic sites, has been established. In some cases, all cuts may not be needed. For example, the occurrence of ectopic sites often starts around the orifices of the PVs and, therefore, it may be sufficient to make the cuts 8, 10 around the PVs. Further, as indicated with the lines 8′ and 10′, the cuts around the PVs may be done along each PV orifice instead of in pairs.

According to the invention, there is provided a possibility of cutting through the heart wall in a new manner. Thus, a similar pattern to the Maze III-pattern should also be achieved according to this new manner. However, as mentioned above, it may not in all cases be required that all cuts of the Maze III-pattern are made.

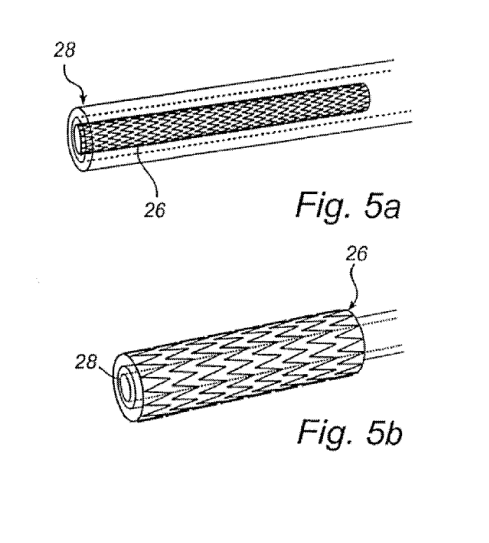

Referring now to FIGS. 4-5, a heart wall tissue lesion creating cutting device 26 according to an embodiment of the invention will be described and the new manner of performing the cuts through the heart wall will be explained. The heart wall tissue lesion creating cutting device 26 (hereinafter called cutting device) is shown in FIG. 4a in a first state, in which the cutting device 26 is tubular and has a first diameter d. The cutting device 26 is shown in FIG. 4b in a second state, in which the cutting device 26 is tubular and has a second diameter D, which is larger than the first diameter d. The cutting device 26 is formed of a shape memory material, which has the ability of memorizing a permanent shape that may significantly differ from a temporary shape. The shape memory material will transfer from its temporary to its memorized, permanent shape as a response to a suitable stimulus. The stimulus may be exposure to a raised temperature, such as a temperature above e.g. 30° C. that may be caused by the body temperature. The stimulus may suitably be combined with the release of a restraining means, which may keep the shape memory material from assuming its permanent shape.

The shape memory material allows designing a cutting device 26 that may be contracted into a small, temporary shape before insertion into a patient. Thus, the cutting device 26 may be inserted in this temporary shape to the heart of a patient through the vascular system. The temporary shape of the cutting device 26 is also flexible, whereby guiding the cutting device 26 through the vascular system is facilitated. This insertion of the cutting device 26 may be performed with well-known percutaneous catheter techniques. This is an unaggressive procedure and may be performed on a beating heart. Thus, the cutting device 26 may readily be positioned at a desired position within the vascular system adjacent heart wall tissue to be treated. The cutting device 26 may then be allowed to transfer to its memorized, permanent shape when inserted to the desired position in a blood vessel.

As shown in FIG. 5a, the cutting device 26 is inserted in its temporary shape in a desired position within a blood vessel 28. As a response to a stimulus, e.g. the body temperature, the cutting device 26 will then strive towards changing its shape and obtaining the permanent shape. The memorized, permanent shape of the cutting device 26 will not fit into the blood vessel 28, whereby the cutting device 26 will force itself through surrounding tissue for obtaining the permanent shape, as shown in FIG. 5b. In this way, the cutting device 26 will first penetrate the vessel wall and thereafter tissue surrounding the blood vessel 28. Tissue cells that are penetrated will be killed, which will start a healing reaction in the body. Where the cutting device 26 is placed in a desired position to change shape through heart wall tissue, cells that are able to transmit electrical signals may thus be killed. The healing process will not restore the ability to transmit electrical signals and, therefore, the cutting device 26 will reduce the ability of transmitting electrical signals through the heart wall. By placing several cutting devices intelligently and designing the permanent shape of the cutting devices 26 accordingly, the cutting devices 26 may penetrate heart wall tissue to create a pattern of cuts corresponding to the Maze III-pattern.

The cutting device may also be spherical and/or globular. This cutting device may present the advantage of being able to affect cutting action in all directions simultaneously.

An example of a shape memory material is Nitinol, which is an alloy composed of nickel (54-60%) and titanium. Small traces of chrome, cobalt, magnesium and iron may also be present. This alloy uses a martensitic phase transition for recovering the permanent shape. Shape memory materials may also be formed of shape memory polymers, wherein the shape-memory effect is based on a glass transition or a melting point. Such shape memory polymers may be produced by forming polymers of materials or combinations of materials having suitable properties. For example, a shape memory polymer may be created of oligo(ε-caprolactone) dimethacrylate combined with n-butyl acrylate. Also, biodegradable or bioresorbable materials may be used for forming these shape memory polymers. Such a biodegradable or bioresorbable material may for example be a polymer, a ceramic, or metallic material.

Biodegradable materials, such as biodegradable polymers, have bonds which are fissionable under physiological conditions. Biodegradableness is the term used if a material decomposes from loss of mechanical properties due to, or within, a biological system. An implant's external form and dimensions may in fact remain intact during the decomposition. This means that a cutting device, which is biodegradable, may also be able to perform cutting action by transforming from a temporary shape to a memory shape. What is meant with respect to degradation time, provided no additional quantifying data is given, is the time it takes for the complete loss of mechanical properties.

A particularly suitable biodegradable material provides for the polymer composite to exhibit a hydrolytically degradable polymer, in particular poly(hydroxy carboxylic acids) or the corresponding copolymers. Hydrolytic degradation has the advantage that the rate at which degradation occurs is independent of the site of implantation since water is present throughout the system.

However, making use of enzymatically degradable polymers is also conceivable in another embodiment. Feasible in particular is that the polymer composite exhibit a biodegradable thermoplastic amorphous polyurethane-copolyester polymer network. Likewise requisite for the chemical composition to the polymer composite for the inventive cutting device is that the polymer composite exhibit a biodegradable elastic polymer network, obtained from crosslinking of oligomer diols with diisocyanate. Having polymer composites be formed as covalent networks based on oligo(ε-caprolactone)dimethacrylate and butylacrylate is a conceivable alternative thereto. For the braiding from which the inventive cutting device is configured, the invention claims both hydrolytically as well as enzymatically degradable polymer composites for the biodegradable polymers. As stated above, hydrolytic degradation has the advantage that the rate at which degradation occurs is independent of implant location. In contrast, local enzyme concentrations vary greatly. Given biodegradable polymers or materials, degradation can thus occur through pure hydrolysis, enigmatically-induced reactions or through a combination thereof.

Typical hydrolyzable chemical bonds for the polymer composites of the cutting device are amide, ester or acetal bonds. Two mechanisms can be noted with respect to the actual degradation. With surface degradation, the hydrolysis of chemical bonds transpires exclusively at the surface. Because of the hydrophobic character, polymer degradation is faster than the water diffusion within the material. This mechanism is seen especially with poly(anhydrides) and poly(orthoesters). As relates to the poly(hydroxy carboxylic acids) particularly significant especially to the present invention, such as poly(lactic acid) or poly(glycol acid), the corresponding copolymers respectively, polymer degradation transpires throughout the entire volume.

The step which determines the rate here is the hydrolytic fission of the bonds since water diffusion in the somewhat hydrophilic polymer matrix occurs at a relatively fast rate. Decisive for the use of biodegradable polymers is that, on the one hand, they degrade at a controlled or variable speed and, on the other, that the products of decomposition are non-toxic.

The concept of polymer material resorption refers to the substance or mass degrading through to the complete removal of a material from the body by way of the natural metabolism. In the case of cutting devices of only one degradable polymer, resorption begins as of that point in time of the complete loss of the mechanical properties. Specification of the resorption time covers the period starting from implantation of the cutting device and running through to the complete elimination of the cutting device.

Among the most important biodegradable synthetic classes of polymers from which, the braiding of the inventive cutting device is advantageously synthesized are; polyesters, such as poly(lactic acid), poly(glycol acid), poly(3-hydroxybutyric acid), poly(4-hydroxyvalerate acid), or poly(ε-caprolactone), or the respective copolymers, polyanhydrides synthesized from dicarboxylic acids, such as, for example, glutar, amber, or sebacic acid, poly(amino acids), or polyamides, such as, for example, poly(serine ester) or poly(aspartic acid).

In summary, it can be stated that shape memory properties play a significant role with respect to said cutting devices, particularly in terms of minimally invasive medicine. Biodegradable cutting devices having shape memory properties are particularly effective in this regard. For example, this type of degradable cutting device can be introduced into the body in compressed (temporary) form through a small incision and once in place, then assume the memory shape relevant to its application after being warmed by the body temperature, as has been described above. The cutting device will then degrade after a given interval of time, thereby doing away with the need for a second operation to remove it.

Based on the known biodegradable polymers, structural elements can be derived for the synthesizing of biodegradable shape memory polymers. In so doing, suitable crosslinks, which fix the permanent form, and network chains, which serve as switching elements, could be selected such that, on the on hand, the switching temperature can be realized through the physiological conditions, and on the other, toxicological problems with respect to any products of decomposition are excluded. Thus, suitable switching segments for biodegradable shape memory polymers can be selected based on the thermal properties of said degradable materials. Of particular interest in this regard is a thermal transition of the switching elements in the temperature range of between room temperature and body temperature. For this transition temperature range, biodegradable polymer segments can be selectively synthesized by varying the stochiometric relationship of the known starting monomers; and the molecular weight of the formed polymers in the range of from approx. 500 to 10000 g/mol.

Suitable polymer segments are e.g. poly(ε-caprolactone)diols with melting temperatures between 46 and 64° C. or amorphous copolyesters based on lactic and glycol acid with glass transition temperatures between 35 and 40° C. The phase transition temperatures hereby; i.e. the melting or glass transition temperature of the polymer switching segments, can be further diminished by their chain length or by degradation of specific end groups. The polymer switching elements thus customized can then be integrated into physical or covalent crosslinked polymer networks, yielding the selectively composed biodegradable shape-memory polymer material.

In one possible embodiment, biodegradable thermoplastic amorphous polyurethane copolyester polymer networks having shape memory properties are used as the material for the cutting device. First, suitable biodegradable star-shaped copolyester polyols are synthesized here based on commercially available dilactide (cyclic lactic acid dimer), diglyocolide (cyclic glycol acid dimer) and trimethylolpropane (functionality F=3) or pentaerythrit (F=4) with glass transition temperatures between 36 and 59° C., which are then crosslinked with commercial trimethylhexa-methylene diisocyanate in forming a biodegradable polyurethane network.

The amorphous polyurethane copolyester polymer networks having shape memory properties as formed have a glass transition temperature T_{K} between 48 and 66° C. and exhibit a modulus of elasticity in extension of between 330 and 600 MPa, a tensile strength respectively of between 18.3 and 34.7 MPa. Heating these networks to approximately 20° C. above this switching temperature yields elastic materials which can be deformed 50-265% into a temporary shape. Cooling down to room temperature occasions the forming of deformed shape memory polymer networks which have a clearly higher modulus of elasticity in extension of from 770 to 5890 Mpa. Upon subsequent reheating to 70° C., the examples of deformed specimens thereby produced retransform back into the permanent shape after approximately 300 seconds. What was ultimately shown, was that polyurethane copolyester polymer networks in an aqueous phosphate buffer decomposed fully at 37° C. over a period of between approximately 80 and 150 days. By optimizing the composition of the biodegradable switching segments, degradable polyurethane copolyester polymer networks having shape memory properties can be produced substantially faster, e.g. within 14 days.

Similar biodegradable elastic shape memory polymer networks can be yielded from crosslinking of oligomer diols with diisocyanate, which have melting temperatures between 38 and 85° C. and which are likewise suitable for the cutting device. Degradableness was also ultimately assessed, whereby for these polymers in an aqueous phosphate buffer at 37° C., a 50% loss of mass was seen after approximately 250 days.

In one embodiment of the cutting device, the braiding is formed from a biodegradable shape memory polymer on covalent networks based on oligo(ε-caprolactone)dimethacrylate and butylacrylate. It has been seen that subsequent implantation, this polymer composite has no negative impacts on the wound healing process. Therefore, the wounds created by the cutting device may heal into a scar tissue, that may prevent unwanted signals to be transmitted. The synthesis of such biodegradable shape memory polymers can follow from n-butylacrylate which, because of the low glass transition temperature of −55° C. for pure poly(n-butylacrylate), can be used as a segment forming component.

Network synthesis ensues through photopolymerization. Based on the molar mass of the macromolecular oligo(ε-caprolactone)dimethacrylate and the content of comonomer n-butylactylate, the switching temperature and the mechanical properties of the covalent network can be controlled. Thus, in an implementation of the manufacturing of a cutting device in an embodiment, the molar mass of the oligo(ε-caprolactone) dimethacrylate varies between 2000 and 10000 g/mol and the n-butylacrylate content between 11 and 90% (by mass). In the case of a polymer network based on a mixture of the low molecular oligo(ε-caprolactone)dimethacrylate at 11% (by mass) of n-butylacrylate, a melting point of 25° C. was realized.

The biodegradable covalent and physical polymer networks, having shape memory effect as described above, can also be used as a matrix for a controlled active substance release. Yet also conceivable would be biodegradable polyurethane multiblock copolymers having shape memory effect based on poly(p-dioxanone) and trimethylhexa-methylene diisocyanate as the diisocyanate.

The combination with the poly(lactid-co-glycolid) or poly(ε-caprolactone) switching segments yields multiblock copolymers having a switching temperature of 37 or 42° C., respectively. The hydrolytic degrading of the polymers shows that the polymers based on poly(ε-caprolactone) degrading at a lesser rate. In a trial on the poly(s-caprolactone) polymers, 50 to 90% of the initial mass was still present after 266 days of hydrolysis while in the case of the poly(lactid-co-glycolid) polymers, 14 to 26% was detectable after only just 210 days.

It can be maintained that biodegradable shape memory polymer networks can be synthesized from a combination of physical or covalent shape memory polymer networks, having biodegradable polymer segments. Selectively choosing the components allows setting optimal parameters for each respective application, such as the mechanical properties, the deformability, the phase transition temperatures and, above all, the switching temperature, as well as the rate of polymer decomposition.

In this respect the invention claims all aforementioned biologically degradable (biodegradable) shape memory polymers as material for the cutting device.

When the cutting device is degraded in a biological environment, such as in a human body, the cutting device will start to elute substances. These substances are parts of the material that the cutting device is made of. If the cutting device for example is made of a polymer, the cutting device will start to release organic substances when the cutting device is degraded in a biological environment, such as a human body. This release may affect the function mechanisms of electrocardial signal transmission, since these function mechanisms are based on physiochemical diffusion effects causing change of pH, change of organic concentrations, and/or change of ionic concentrations, which physiochemical diffusion effects may be affected by the substances released by the cutting device when the cutting device is degraded. Myocardial muscle cells presents activation potentials and charging conditions in respect of operation status. These potentials and conditions are dependant on their electrolyte environment and which substances are present in the vicinity of said muscle cells. Thereby cell membrane function may be affected by a change in pH, resulting from release of substances from the cutting device during degradation. Change in organic concentrations may result in chelating effects in respect of ions, hydrophobic effects in the vicinity of the cell membrane, and/or pharmacological effects on cell membrane function, when substances are released from the cutting device during degradation. Organic release, even if the release is of carbon dioxide and water, may influence membrane potential and function. This may for example be achieved by changing pH, changing the ion activity of specific ions needed for specific functions, such as Na or Ca. Release of oxalic acid anions may for example have a chelating effect on Ca. This may lead to degenerative effects on the membrane of muscle cells, such as the myocardiac cells and/or postsynaptic membranes. Also ceramic or metallic biodegradable materials may release ions. These ions may for example affect myocardial cell activity, such as through increase in Li and/or Mg concentrations causing short and/or long term changes in electrical signal transmission. According to an article of Fleed and Ferrans, it has been shown that especially Li, Mg, Ni, Co, and V may have these effects.

The release of substances from the cutting device itself may be combined, which combination affects myocardial signal propagation, may be used as a synergetic effect when treating atrial fibrillation. The effect of the cutting device may therefore be enhanced by achieving a more immediate effect than only the cutting action.

A change in pH may result in an increase of local inflammation in myocardial muscle tissue. Fast resorbing polymers, such as poly(glycolic) acid, may have this effect on tissue. Also, more effective tissue reaction are known from the testing of resorbable copolymers, since they can degrade faster than their higher crystalline homopolymers from which they are made of. Examples of such copolymers are lactide/caprolactone copolymers. In one embodiment this effect is taken advantage of, since an increase in inflammation will result in larger area of scar tissue. A larger area of scar tissue will increase the effect of isolating signal transmission. In this respect a polymer is designed as a resorbable polymer with the aim to release non-toxic and already known monomers by hydrolysis, such as glycolic acid or perhaps oxalic acid. Resorbable classes of ceramics, since they are build from metal oxides, may be designed to release ions causing the pH to change towards alkalic environment. Most resorbable ceramics are composed based on hydroxyapatite (Ca-phosphate salts). Hydroxyapathite is from the chemical point of view a buffer which can be composed towards alkalic or acidic behaviour. It will also be possible, while being inside the scope of the present invention to fill a resorbable system with anhydrides of acids or bases. The release of known substances, such as monomers or ions, where the safe metabolism, and the non toxic behaviour, is already known, might be advantageous. Thus, to achieve an increase of local inflammation in myocardial muscle tissue, the cutting device may be manufactured of such polymers, with these advantages and possibilities.

In this way, the cutting device 26 may be designed such that it will be degraded or absorbed by the body after it has performed its change of shape. For example, a polylactic acid polymer and/or a polyglycolic acid polymer, poly(ε-caprolactone) or polydioxanone, according to above, may be used for forming a shape memory polymer that is biodegradable. A special feature of the resorbable shape memory polymers is that these will disappear from the tissue after having had its function, limiting potential negative effects of otherwise remaining polymer or Nitinol materials, such as perforations and damage to other adjacent tissues, like lungs, oesophagus and great vessels like the aorta.

The cutting device 26 may be tubular in both its temporary shape and its permanent shape, as shown in FIGS. 4-5. However, the shape memory may be used for bringing the cutting device 26 between any shapes. Some examples of shapes that are at least not entirely tubular are for example globular, spiral shaped, cork screw shaped, and shapes adapted to fit or be arranged in a specific area, such as in the heart. This specific area in the heart may be an atrium or a ventricle. First picturing said tissue or area, and subsequently adapting the cutting device according to the obtained picture may for example perform the adaptation of the cutting device. The shape of the cutting device 26 in its first state is preferably compact to facilitate insertion of the cutting device 26 through the vascular system. Thus, a tubular shape is suitable, but other shapes, according to above, may be just as suitable. Further, the shape of the cutting device 26 in its second state is designed such that the change of shape will provide penetration of specific heart tissue in order to block propagation of undesired electrical signals. Also, the shape of the cutting device 26 in its second state may be adjusted for fixing the cutting device 26 to its desired position within the body.

The cutting device 26 may be constructed of a net; i.e. its shape may comprise meshes or loops. This implies that a solid surface need not penetrate tissue, whereby the penetration through tissue and the forming of different shapes of the cutting device 26 will be facilitated.

The edges of the cutting device 26 facing the tissue to be penetrated may be made especially sharp to increase its effectiveness, as illustrated in FIG. 4c. Another feature is to cover the surface towards the tissue to be penetrated with drugs that increase the cutting effect or prohibit the thickening of the wall of the vessel in which the device is inserted. Examples of such drugs are ciclosporin, taxiferol, rapamycin, tacrolimus, alcohol, glutaraldehyde, formaldehyde, and proteolytic enzymes like collagenase. Collagenase is effective in breaking down tissue and especially fibrin tissue, which is otherwise difficult to penetrate. Therefore, covering the surface of the cutting device 26 with collagenase would particularly speed up the process of penetrating tissue. The drugs are attached to the surface of the cutting device 26 according to well-known methods of attaching drugs to medical devices. One such method is embedding drugs into or under layers of polymers, which cover the surface. Of course, other methods may be used. Similarly, drugs preventing thrombosis and increasing in-growth of endothelium on the endothelial surface after penetration of the cutting device 26 may be attached to the cutting device 26. Such drugs would be e.g. Endothelium Growth Factor, and Heparin. Also, other drugs designed to treat arrhythmias may be attached to the cutting device surface. Such drugs are e.g. amiodarone and sotalol.

Since the cutting device according to the present invention is manufactured of a biodegradable material, it is also possible to integrate the drug, such as those mentioned above, in the biodegradable material. Thus, as the biodegradable material degrades in a biological environment, the drug is eluted continuously. In one embodiment a drug, or a plurality of drugs, may be integrated as sheets in the biodegradable material. This embodiment provides the possibility to elute a drug during separated time intervals, or elute different drugs at different points of time. In still another embodiment a drug, or a plurality of drugs, are integrated homogenously in the biodegradable material.

It is of course possible to have a drug, or plurality of drugs, integrated in the biodegradable material of the cutting device, and also coat the surface of the cutting device with a coating of a drug, or plurality of drugs. This kind of coating may cover the whole cutting device or only a part of the cutting device, such as a cutting edge.

In still another embodiment the cutting device comprising a drug, or a plurality of drugs, is coated with a biodegradable material not containing any drug or drugs. Thus, it will be possible to regulate the point of time for the elution of said drug or drugs. This point of time will be regulated by varying the thickness of the coating not containing any drug or drugs. When the coating has been degraded the material containing the drug or drugs will be uncovered, and since also this material is biodegradable the material will start to degrade with accompanying elution of said drug or drugs.

It may for example be possible to integrate in the cutting device one drug, which is active on collagen or elastin, while another drug may be included to act on muscle tissue.

Preferably, the inside of the cutting device 26 inserted into a blood vessel will be in contact with the blood stream inside the blood vessel. Such inside surface of the cutting device 26 may as well be covered with antithrombotic drugs. Such drugs would be e.g. Heparin, Klopidogrel, Enoxaparin, Ticlopidin, Abciximab, and Tirofiban. It is also possible to integrate these drugs in the biodegradable material in the different ways described above.

Another way to increase the effectiveness of the cutting device 26 is to attach a metallic part of the cutting device 26 to electrical currency, which would provide a heating of the cutting device 26. Thereby, tissue may also be killed by this heating, enhancing the effect of the cutting device 26. Further, the force driving the change of shape will also be increased, speeding up the shape change of the cutting device.

Moreover, other design parameters of tissue cutting devices may be chosen according to patient specific anatomy. Such design parameters are for instance wire thickness distribution, connection points, fastening elements such as hooks, bistable sections or characteristics, material choice, implementation of drug delivery sections, timing design of cutting action, etc. as described in co-pending patent applications concurrently filed by same applicant as present application, which hereby are incorporated by reference herein in their entirety.

Hereinafter, some potential uses of the present invention are described:

A method for treatment of disorders in the heart rhythm regulation system comprising

inserting a tissue cutting device in a temporary delivery shape through the vascular system into a body vessel adjacent to the heart and/or into the heart;

changing shape of the tissue cutting device, from said temporary delivery shape via an expanded delivered shape to a further expanded shape, extending at least beyond an outer surface of said tissue, thereby

creating cutting action configured for cutting said heart tissue and/or said body vessel, thereby

reducing undesired signal transmission in a heart tissue by isolating ectopic sites thereof by cutting said tissue by means of the tissue cutting device configured therefore, and

biodegrading the tissue cutting device during or after said changing shape of the tissue cutting device from said expanded delivered shape to said further expanded shape.

The method according to the above, said method comprising inserting a tissue cutting device through the vascular system to a desired position in a body vessel, and providing a change of shape of the tissue cutting device at said desired position to penetrate heart tissue adjacent said body vessel.

The method according to above, wherein said tissue cutting device is inserted into a desired position in the coronary sinus, in any of the pulmonary veins, in the superior vena cava, in the inferior vena cava, or in the left or right atrial appendage.

The method according to above, further comprising inserting another tissue cutting device to another of the desired positions.

The method according to above, further comprising inserting a tissue cutting device into each of the desired positions.

The method according to above, further comprising restraining the tissue cutting device in an insertion shape during the inserting of the tissue cutting device.

The method according to above, wherein the restraining comprises keeping the tissue cutting device inside a tube.

The method according to above, wherein the restraining comprises cooling the tissue cutting device.

The method according to above, further comprising releasing a restrain on the tissue cutting device when it has been inserted into the desired position for allowing said change of the shape of the tissue cutting device.

The method according to above, wherein said biodegrading the tissue cutting device comprises hydrolytically or enzymatically degrading said tissue cutting device.

It should be emphasized that the preferred embodiments described herein is in no way limiting and that many alternative embodiments are possible within the scope of protection defined by the appended claims.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A tissue cutting device configured to create lesions to reduce undesired signal transmission in a heart tissue by isolating ectopic sites thereof by cutting said tissue,
wherein the device is structured and arranged to be inserted in a temporary delivery shape through the vascular system into a body vessel adjacent to the heart and/or into the heart and to be subsequently subjected to a change of shape, from said temporary delivery shape via an expanded delivered shape to a further expanded shape, extending at least beyond an outer surface of said tissue, in order to create cutting action configured for cutting said heart tissue and/or said body vessel in order to create wounds that heal into a scar tissue that prevents said undesired signal transmission,
wherein said tissue cutting device is of a biodegradable material, such that said tissue cutting device is configured to biodegrade during or after said changing shape of the tissue cutting device from said expanded delivered shape to said further expanded shape, whereby said biodegradable material, in use, enhances said cutting action.

2. The tissue cutting device according to claim 1, wherein said biodegradable material is a bioresorbable material.

3. The tissue cutting device according to claim 1, wherein said biodegradable material is a biodegradable polymer, wherein said tissue cutting device is configured to, in use, start to release at least one organic substance when said tissue cutting device being degraded in a biological environment.

4. The tissue cutting device according to claim 3, wherein said tissue cutting device is configured to affect electrocardial signal transmission, by said organic substances released by the tissue cutting device when the tissue cutting device in use is degraded.

5. The tissue cutting device according to claim 4, wherein said organic substances in use of the tissue cutting device released from said biodegradable polymer cause an inflammation in said heart tissue to provide said enhanced cutting action.

6. The tissue cutting device according to claim 5 wherein, said organic substances released by the tissue cutting device when the tissue cutting device in use is degraded provide a change in pH, such that local inflammation in myocardial muscle tissue is increased.

7. The tissue cutting device according to claim 5, wherein said biodegradable polymer is a fast resorbing polymer.

8. The tissue cutting device according to claim 7, wherein said fast resorbing polymer is a polylactic acid polymer or a poly(glycolic) acid polymer.

9. The tissue cutting device according to claim 5, wherein said biodegradable polymer is a resorbable copolymer that degrade faster than their higher crystalline homopolymers from which they are made of.

10. The tissue cutting device according to claim 9, wherein said resorbable copolymer is a lactide/caprolactone copolymer.

11. The tissue cutting device according to claim 2, wherein said bioresorbable material is a resorbable ceramic material designed to release ions causing the pH to change towards an alkalic environment.

12. The tissue cutting device according to claim 11, wherein resorbable ceramic material is Hydroxyapathite.

13. The tissue cutting device according to claim 1, wherein said biodegradable material is a hydrolytically degradable material.

14. The tissue cutting device according to claim 13, wherein said hydrolytically degradable material is a poly(hydroxyl carboxylic acid), a poly(anhydride) or a poly(orthoester).

15. The tissue cutting device according to claim 14, wherein said poly(hydroxyl carboxylic acid) is poly(lactic acid) or poly(glycol acid), or copolymers thereof.

16. The tissue cutting device according to claim 1, wherein said biodegradable material is an enzymatically degradable material.

17. The tissue cutting device according to claim 16, wherein said enzymatically degradable material is selected from oligo(ε-caprolactone) dimethacrylate or butylacrylate.

18. The tissue cutting device according to claim 1, wherein said biodegradable material is selected from the group consisting of polyesters, such as poly(lactic acid), polytglycol acid), poly(3-hydroxybutyric acid), poly(4-hydroxyvalerate acid), or poly(ε-caprolactone), or the respective copolymers, polyanhydrides synthesized from dicarboxylic acids, such as, for example, glutar, amber, or sebacic acid, poly(amino acids), or polyamides, such as, for example, poly(serine ester) or poly(aspartic acid).

19. The tissue cutting device according to claim 1, wherein said biodegradable material is a ceramic or metallic material.

20. The tissue cutting device according to claim 19, wherein said metallic material comprises Li, Mg, Ni, Co, and/or V.

21. The tissue cutting device according to claim 1, wherein said biodegradable material, in use, elutes at least one substance when being degraded in a biological environment.

22. The tissue cutting device according to claim 21, wherein said at least one substance is an organic substance, or an ion.

23. The tissue cutting device according to claim 22, wherein said ion is selected from the group comprising Li, Mg, Ni, Co, and/or V.

24. The tissue cutting device according to claim 1, comprising at least one drug.

25. The tissue cutting device according to claim 24, wherein said at least one drug is comprised in a coating or as layers within said tissue cutting device.

28. The tissue cutting device according to claim 24, wherein said at least one drug is a drug that increases said cutting effect, such as ciclosporin, taxiferol, rapamycin, tacrolimus, alcohol, glutaraldehyde, formaldehyde, and proteolytic enzymes.

27. The tissue cutting device according to claim 26, wherein proteolytic enzyme is collagenase, whereby said cutting effect is accelerated.

28. The tissue cutting device according to claim 24, comprising a coating of a non-drug biodegradable containing material.

29. The tissue cutting device according to claim 1, wherein the device is structured and arranged to be inserted into a body vessel and to subsequently change shape, wherein the device is structured and arranged to change shape to extend at least partly outside the perimeter or orifice of an outer wall of said vessel in said further expanded shape.

30. A kit of shape-changing cutting devices according to claim 1 for treatment of disorders in the heart rhythm regulation system, said kit comprising:
a plurality of said shape-changing tissue cutting devices, which each has a first delivery and a second delivered state, wherein each tissue cutting device in the first state has such dimensions as to be insertable to a desired position within the vascular system, and wherein each tissue cutting device is capable of changing shape to substantially the second state when located at said desired position, which strives to a diameter that is larger than the diameter of the vessel at the desired position, whereby the tissue cutting device will become embedded into the tissue surrounding the vessel at the desired position and destroy the tissue in order to prevent it from transmitting electrical signals,
wherein at least one of the shape-changing devices is adapted to be inserted to a desired position at the orifice of a pulmonary vein in the heart, and at least one of the shape-changing devices is adapted to be inserted to a desired position in the coronary sinus, and wherein said tissue cutting devices are of a biodegradable material, such that said plurality of tissue cutting devices, in use, create a pattern of cuts corresponding to the Maze III-pattern for said treatment of disorders in the heart rhythm regulation system.

31. A method for treatment of disorders in the heart rhythm regulation system comprising
inserting a tissue cutting device in a temporary delivery shape through the vascular system into a body vessel adjacent to the heart and/or into the heart;
changing shape of the tissue cutting device, from said temporary delivery shape via an expanded delivered shape to a further expanded shape, extending at least beyond an outer surface of said tissue, thereby creating cutting action configured for cutting said heart tissue and/or said body vessel, thereby
reducing undesired signal transmission in a heart tissue by isolating ectopic sites thereof by cutting said tissue by means of the tissue cutting device configured therefore, and
biodegrading the tissue cutting device during or after said changing shape of the tissue cutting device from said expanded delivered shape to said further expanded shape.

32. The method according to claim 31, comprising inserting a tissue cutting device through the vascular system to a desired position in a body vessel, and providing a change of shape of the tissue cutting device at said desired position to penetrate heart tissue adjacent said body vessel.

33. The method according to claim 31, wherein said tissue cutting device is inserted into a desired position in the coronary sinus, in any of the pulmonary veins, in the superior vena cava, in the inferior vena cava, or in the left or right atrial appendage.

34. The method according to claim 31, further comprising inserting at least another tissue cutting device to another of a plurality of desired positions.

35. The method according to claim 34, further comprising inserting a tissue cutting device into each of the plurality of desired positions.

36. The method according to claim 31, further comprising restraining the tissue cutting device in an insertion shape during the inserting of the tissue cutting device.

37. The method according to claim 36, wherein the restraining comprises keeping the tissue cutting device inside a tube.

38. The method according to claim 36, wherein the restraining comprises cooling the tissue cutting device.

39. The method according to claim 36, further comprising releasing a restrain on the tissue cutting device when it has been inserted into the desired position for allowing said change of the shape of the tissue cutting device.

40. The method according to claim 31, wherein said biodegrading the tissue cutting device comprises hydrolytically or enzymatically degrading said tissue cutting device, enhancing said cutting action.

41. The method according to claim 31, comprising eluting at least one drug from said tissue cutting device, accelerating said cutting action.
